(19) United States
(12) Patent Application Publication
(10) Pub. No.: US 2024/0180753 A1
(43) Pub. Date: Jun. 6, 2024

(54) COMPRESSION APPARATUS

(71) Applicant: MAS Innovation (Private) Limited, Battaramulla (LK)

(72) Inventors: Angelo KARUNARATNE, Battaramulla (LK); Mihan ZAIN, Battaramulla (LK); Shahir HANIFFA, Battaramulla (LK); Udara Pulajith WEERAMUNDA SAYAKKARA, Battaramulla (LK)

(21) Appl. No.: 18/285,933

(22) PCT Filed: Apr. 8, 2022
(86) PCT No.: PCT/SG2022/050204

§ 371 Date: Oct. 6, 2023

(30) Foreign Application Priority Data
Apr. 8, 2021 (GB) 2105008.3

(51) Int. Cl. A61F 13/08

(52) U.S. Cl. CPC A61F 13/085

(57) ABSTRACT

The present disclosure generally relates to a compression apparatus (100) for applying compression to a body part (200). The compression apparatus (100) comprises: a wearable device (102) wearable on the body part (200), the wearable device (102) comprising a compression portion (104) for applying main compression to the body part (200): and a tensioning device (120) attached to the wearable device (102) and wearable on the body part (200), the tensioning device (120) comprising: an inelastic portion (124) aligned to the compression portion (104) and for arranging on the body part (200); and an elastic portion (122) attached to one end of the inelastic portion (124). When the wearable device (102) and tensioning device (120) are worn on the body part (200), the elastic portion (122) is attachable to an opposing end of the inelastic portion (124) such that the elastic portion (122) is stretched and the tensioning device (120) is tightened around the compression portion (104), thereby causing the compression portion (104) to apply base compression to the body part (200); and wherein the main compression adds to the base compression to increase overall compression applied to the body part, the inelastic portion facilitating even distribution of the overall compression on the body part.

### CROSS REFERENCE TO RELATED APPLICATION(S)

The present disclosure claims the benefit of United Kingdom Patent Application No. 2105008.3 filed on 8 Apr. 2021, which is incorporated in its entirety by reference herein.

### TECHNICAL FIELD

The present disclosure generally relates to a compression apparatus. More particularly, the present disclosure describes various embodiments of a compression apparatus for applying compression to a body part of a user.

### BACKGROUND

Arteries carry blood from the heart out to other parts of the body, such as the limbs, and veins carry blood back to the heart, and valves in the veins stop the blood from flowing backward. Compression therapy benefits people with circulation issues, especially those who often engage in sports activities, by improving venous return through effective muscle pump action. Compression devices can improve venous return through application of graduated compression to body parts.

These compression devices can provide active compression and/or static compression to the body part. However, active compression devices require electrical power to operate and cease to apply any compression in absence of power. This can create issues for users who need the compression to alleviate pain or aches that commonly result after sports activities, but who find themselves in a situation where the active compression device has inadequate power supply. Some of these compression devices do not provide adequate compression and/or not suitable for use in various body parts, such as legs, arms, torso, etc.

Therefore, in order to address or alleviate at least one of the aforementioned problems or disadvantages, there is a need to provide an improved compression apparatus.

### SUMMARY

According to an aspect of the present disclosure, there is a compression apparatus for applying compression to a body part of a user, the compression apparatus comprising: a wearable device wearable on the body part, the wearable device comprising a compression portion for applying main compression to the body part; and a tensioning device attached to the wearable device and wearable on the body part, the tensioning device comprising: an inelastic portion aligned to the compression portion and for arranging on the body part; and an elastic portion attached to one end of the inelastic portion. When the wearable device and tensioning device are worn on the body part, the elastic portion is attachable to an opposing end of the inelastic portion such that the elastic portion is stretched and the tensioning device is tightened around the compression portion, thereby causing the compression portion to apply base compression to the body part; and wherein the main compression adds to the base compression to increase overall compression applied to the body part, the inelastic portion facilitating even distribution of the overall compression on the body part.

A compression apparatus according to the present disclosure is thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIG. 1 is an illustration of a compression apparatus in open form, in accordance with embodiments of the present disclosure.

FIG. 2A to FIG. 2D are illustrations of the compression apparatus in open form together with a body part, in accordance with embodiments of the present disclosure.

FIG. 3A and FIG. 3B are illustrations of the compression apparatus in closed form, in accordance with embodiments of the present disclosure.

FIG. 4A to FIG. 4C are illustrations of the compression apparatus in closed form and worn on the body part, in accordance with embodiments of the present disclosure.

FIG. 5 show results of tests performed on the compression apparatus.

FIG. 6 is a cross-sectional illustration of the compression apparatus with the body part, in accordance with embodiments of the present disclosure.

FIG. 7A and FIG. 7B are additional illustrations of the compression apparatus in open form, in accordance with embodiments of the present disclosure.

### DETAILED DESCRIPTION

In the present disclosure, depiction of a given element or consideration or use of a particular element number in a particular figure or a reference thereto in corresponding descriptive material can encompass the same, an equivalent, or an analogous element or element number identified in another figure or descriptive material associated therewith. The use of “/” in a figure or associated text is understood to mean “and/or” unless otherwise indicated. The recitation of a particular numerical value or value range herein is understood to include or be a recitation of an approximate numerical value or value range. The term “set” is defined as a non-empty finite organization of elements that mathematically exhibits a cardinality of at least one (e.g. a set as defined herein can correspond to a unit, singlet, or single-element set, or a multiple-element set), in accordance with known mathematical definitions.

For purposes of brevity and clarity, descriptions of embodiments of the present disclosure are directed to a compression apparatus in accordance with the drawings.

While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail so as to not unnecessarily obscure aspects of the embodiments of the present disclosure.

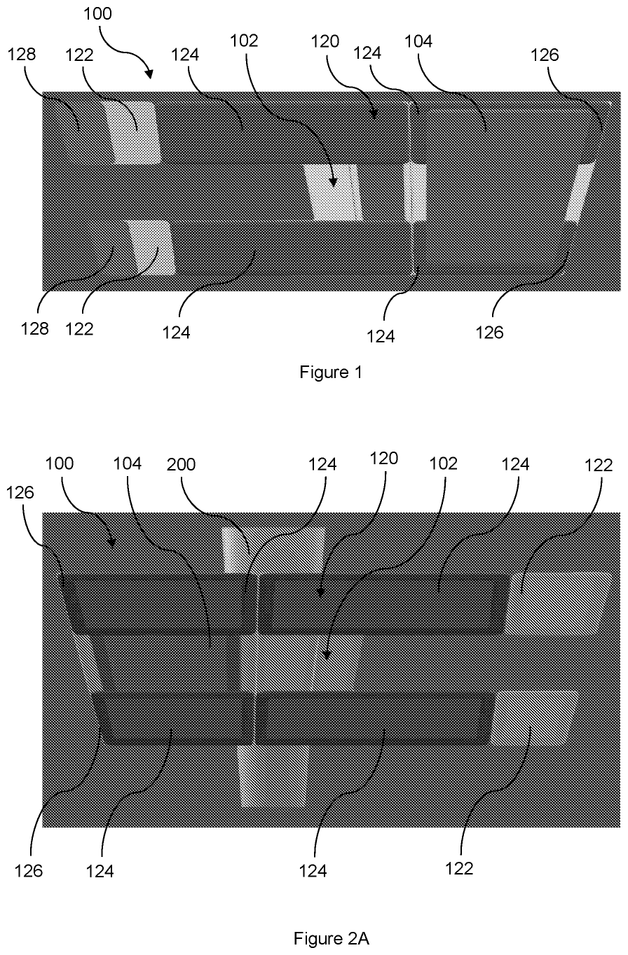

In representative or exemplary embodiments of the present disclosure, there is a compression apparatus 100 for applying compression pressure to a body part 200 of a user. In some embodiments, this body part 200 is an extremity such as a limb of the user, as illustrated in FIG. 1. The extremity or limb may be a lower limb of the user, such as the leg or a part of the leg. In some other embodiments, the body part 200 may be the torso, neck, or navel area (or portions thereof) of the user. In yet some other embodiments, the body part 200 may be distal parts of the user's extremities, such as the digits or fingers.

The compression apparatus 100 includes a wearable device or portion 102, such as a garment, for the user to wear on the body part 200. For example, the wearable device 102 may be in the form of a sleeve like a compression sock shaped to be worn on the lower leg of the user, such as at the calf region. The wearable device 102 may be in the form of a compression wrap which the user can wrap around the leg or other parts like the neck or torso. Such a compression wrap may include fastening portions with fastening surfaces such as hook-and-loop/touch fasteners which allow the wearable device 102 to be fastened and tightened around the body part 200.

In some embodiments, the compression apparatus 100 is capable of active compression and may include compression or actuation devices, such as inflatable bladders and soft actuators, for compressing against the body part 200. In some embodiments, the compression apparatus 100 is capable of passive compression, such as if the wearable device 102 is in the form of a compression sock. In some embodiments, the compression apparatus 100 is capable of both active and passive compression.

The wearable device 102 includes a compression portion 104 for applying main compression to the body part 200. The main compression may include active and/or passive compression. In some embodiments, the compression portion 104 is configured to hold a compression or actuation device. More generally, the wearable device 102 may include a set of one or more compression portions 104 for holding one or more compression devices. In some embodiments, the compression portion 104 includes an inflatable bladder for actively compressing against the body part 200. The inflatable bladder is fluidically connected to an inflation device, such as a pump, for inflation and deflation. Inflation of the inflatable bladder actuates and expands the compression portion 104, enabling the compression portion 104 to compress against the body part 200 where the wearable device 102 is worn.

In some embodiments, the wearable device 102 includes a plurality of compression portions 104. For example, the wearable device 102 has a plurality of inflatable bladders arranged at the compression portions 104 to apply compression pressure to various portions of the body part 200. The inflatable bladders may be configured to apply varying compression pressure to different portions of the body part 200. For example, the wearable device 102 can be worn on the body part 200 such as a limb of the user, or more specifically the lower leg (below the knee), and varying compression pressure can be applied to different portions of the lower leg. When the wearable device 102 is worn on the user's lower leg, the inflatable bladders may target the compression pressure at the upper calf region, lower calf region, and ankle region.

The wearable device 102, particularly the compression portion 104, may be formed of a stretchable material to cater for the compression, such as inflation and deflation of an inflatable bladder. The stretchable material may include a suitable fabric material. The stretchable material provides for adjustability to allow the wearable device 102 to fit closely to the body part 200.

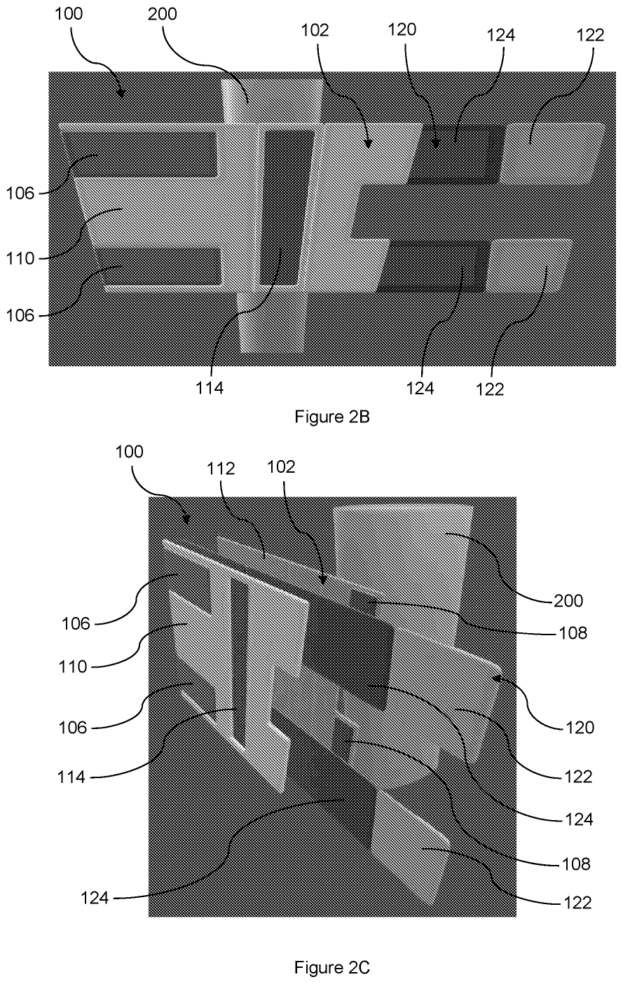

The compression apparatus 100 further includes a tensioning device 120 attached to the wearable device 102 and aligned to the compression portion 104. As shown in FIG. 1 and FIG. 2A, the tension device 120 is arranged such that it overlays the compression portion 104. This enables the tension device 120 to cooperate with the compression portion 104 when the compression portion 104 compresses against the body part 200. More specifically, when the wearable device 102 and tensioning device 120 are worn on the body part 200, the tensioning device 120 is configured to tighten around the compression portion 104, thereby causing the compression portion 104 to apply base compression to the body part 200.

By tightening the tension device 120 so that it is in tension, a base level of compression can be applied to the body part 200 before operating any compression or actuation device, such as inflating an inflatable bladder. This base level of compression pressure may be called pretension pressure or initial pressure. Thus, when the user wears the wearable device 102 and tightens the tensioning device 120, the user is able to achieve at least this base compression on the body part 200. The base compression complements the main compression from the compression device, such that the main compression adds to the base compression to increase the overall compression applied to the body part 200.

For example, when there is positive main compression, it combines with the base compression to increase the overall compression applied to the body part 200. The base compression sets the starting pressure level for the main compression. When the main compression stops, there is still the base compression which applies some level of pretension pressure to the body part 200. This advantageously enables the user to continue with compression therapy in situations where the compression or actuation devices fail to operate. For example, there may be inadequate power supply to power any compression or actuation devices of the compression apparatus 100, such as inflation devices or pumps.

In some embodiments, the tensioning device 120 is removably attached to the wearable device 102. This allows the tensioning device 120 to be detached, such as for replacement due to wear and tear or if it becomes damaged. In some embodiments, the tensioning device 120 is integrated with the wearable device 102 and cannot be detached from the wearable device 102. The tensioning device 120 can be integrally or permanently attached to the wearable device 102 during manufacturing of the compression apparatus 100.

In many embodiments as shown in FIG. 1, the tensioning device 120 includes an elastic portion 122 and an inelastic portion 124. The inelastic portion 124 may be formed in one or more sections. The elastic portion 122 is attached to one end of the inelastic portion 124 such that the tensioning device 120 is in the form of a continuous strap. For example, the elastic portion 122 is stitched or bonded to the inelastic portion 124. Alternatively, the elastic portion 122 may be knitted to the inelastic portion 124 to achieve smooth transition across inelastic and elastic properties.

The elastic portion 122 is attachable to an opposing end of the inelastic portion 124 to tighten the tensioning device 120 around the compression portion 104. The tensioning device 120 is arranged with respect to the wearable device 102 such that the inelastic portion 124 is aligned to the compression portion 104. When the wearable device 102 is worn on the body part 200, the elastic portion 122 is attached to the opposing end of the inelastic portion 124, such that the elastic portion 122 is stretched and the tensioning device 120 is tightened around the compression portion 104. This causes the compression portion 104 to apply the base compression to the body part 200.

The inelastic portion 124 is aligned to the compression portion 104 such that when the compression portion 104 applies the main compression to the body part 200, such as by inflating the bladder for active compression, the inelastic portion 124 does not absorb the compression pressure which would reduce the amount of compression pressure applied to the body part 200. The alignment of the inelastic portion 124 to the compression portion 104 advantageously stabilizes the overall compression (including both main and base compressions) applied to the body part 200, thus facilitating even distribution of the overall compression on the body part 200. Additionally, by providing the base compression, the amount of active compression can be reduced. This reduces the power required for the active compression and mitigates energy loss, thereby improving management of energy usage.

The tensioning device 120 may be attached to the wearable device 102 by attaching the opposing end of the inelastic portion 124 to the wearable device 102. In one example, the opposing end is bonded to the wearable device 102 using suitable adhesive or bonding means, such as adhesive glue or ultrasonic bonding. In another example as shown in FIG. 1 and FIG. 2A, the opposing end is knitted, sewn, or stitched 126 to the wearable device 102. The other edges of the inelastic portion 124 may not be attached to the wearable device 102 so that the tensioning device 120 remains free for the user to pull around the body part 200. It will be appreciated that the inelastic portion 124 can be attached to the wearable device 102 using various arrangements and means as will be readily known to the skilled person.

The elastic portion 122 is made of or includes an elastic or stretchable material. In some embodiments, the elastic material includes an elastomeric fabric material. The elastomeric fabric material may include, but is not limited to, rubber, polybutadiene, polyisobutylene, polyurethane, or any combination thereof. The inelastic portion 124 is made of or includes an inelastic material. The inelastic material may be a smooth fabric material having a low coefficient of friction, such as taffeta or other silk-like material.

The weight of taffeta fabric can vary depending on the composition of the taffeta fabric. For example, some taffeta fabrics are lightweight, such as nylon taffeta and polyester taffeta, which are comparable to other man-made fabrics such as rayon, viscose, and parachute fabric. Some taffeta fabrics are medium weight and they may contain medium weight cottons such as batiste, broadcloth, calico, cambric, chambray, and muslin. Some taffeta fabrics are heavy weight, such as heavy weight wool taffeta which may include burlap, butcher's linen, canvas, and tweed.

To use the compression apparatus 100, the user first wears the wearable device 102 on the body part 200 and secures it using suitable fastening elements, as described below. The user then adjusts the tensioning device 120 around the body part 200 and pulls the elastic portion 122. This stretches the elastic portion 122 and tensions the inelastic portion 124 as well, thereby tightening the tensioning device 120 around the compression portion 104. The user then attaches the stretched elastic portion 122 to the opposing end of the inelastic portion 124. The tightened tensioning device 120 causes the compression portion 104 to apply the base compression to the body part 200. In addition, the user can adjust the level of base compression, such as to a more comfortable level for the user or to a desired level for compression therapy, by adjusting the stretch of the elastic portion 122. Notably, a larger extension of the elastic portion 122 would increase the base compression.

As shown in FIG. 2B to FIG. 2D, the wearable device 102 may include fastening areas 106 and fastening elements 108 that are mutually engageable with each other to facilitate wearing and securing of the wearable device 102 on the body part 200. The fastening elements 108 may be provided on the outer tabs of the wearable device 102. The fastening areas 106 and fastening elements 108 may be engageable in a similar way as touch fasteners or hook-and-loop fasteners. Some other non-limiting examples of the fastening engagement may be by way of hook-and-eye fasteners, magnetic fasteners or strips, snap fasteners or buttons, fastening buckles, and fastening straps.

The tensioning device 120 may include a single strap having the elastic portion 122 and inelastic portion 124. Alternatively, such as shown in FIG. 2A to FIG. 2D, the tensioning device 120 may include a plurality of straps each having the elastic portion 122 and inelastic portion 124. The plurality of straps allows the user to adjust the stretch of each elastic portion 122 and adjust the tension of the inelastic portion 124 accordingly. As the plurality of inelastic portions 124 are arranged at various portions of the body part 200, the user can customize the level of base compression at different portions of the body part 200.

In some embodiments, the elastic portion 122 and the inelastic portion 124 include mutually engageable fastening elements. For example as shown in FIG. 1, the elastic portion 122 includes or is attached to fastening elements 128 that are attachable to the opposing end of the inelastic portion 124. In one embodiment, the fastening elements 128 are positioned near the fastening elements 108 of the wearable device 102 and attachable to the inelastic portion 124 at the fastening areas 106. Thus, after the user wears the wearable device 102 and fastens the fastening elements 108 to the fastening areas 106, the user tightens the tensioning device 120 and fastens the fastening elements 128 to the fastening areas 106. In another embodiment, the inelastic portion 124 has corresponding fastening areas at the opposing end for fastening with the fastening elements 128. In another embodiment, the fastening elements 108 and 128 are integrated with each other so that the fastening elements 108 and 128 can be together fastened to the fastening areas 106. This makes it easier for the user to use the compression apparatus 100 as the user can wear the wearable device 102 and tighten the tensioning device 120 in a single movement.

As shown in FIG. 2B to FIG. 2D, the fastening areas 106 of the wearable device 102 may be formed on an outer layer 110 of the wearable device 102. The wearable device 102 may further include a skin-contact layer 112 for interfacing between the wearable device 102 and the body part 200. The fastening elements 108 of the wearable device 102 may be formed on the outer layer 110 or extending outwardly from the skin-contact layer 112. The skin-contact layer 112 is arranged such that, when the wearable device 102 is worn on the body part 200, the skin-contact layer 112 is in physical contact with the skin of the body part 200. Due to this skin contact and to improve comfort to the user, the skin-contact layer 112 may be made of a suitable material and has a smooth surface finish, such as a smooth fabric material, to reduce friction between the wearable device 102 and the body part 200. In addition to having a low coefficient of friction, the material of the skin-contact layer 112 may have other beneficial properties such as breathability and moisture wicking.

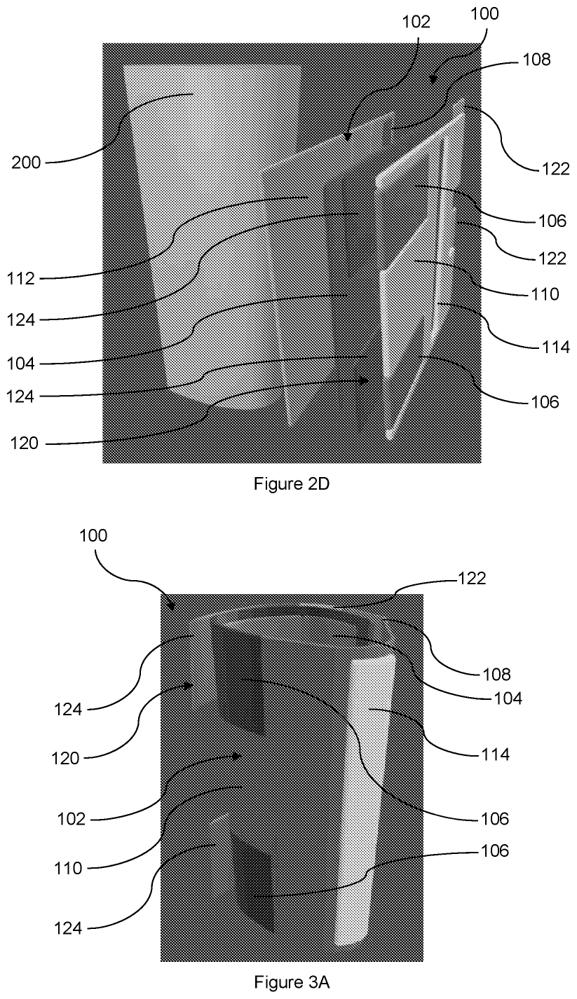

As shown in FIG. 3A and FIG. 3B, the outer layer 110 of the wearable device 102 may include or may be configured to hold a control module 114 for controlling the main compression applied by the compression portion 104. For example, the control module 114 is configured to control actuation devices, such as inflation devices and soft actuators. More specifically, the control module 114 may be configured to control the inflation devices or pumps to inflate and deflate a bladder disposed at the compression portion 104. In some embodiments, the wearable device 102 has a plurality of compression portions 104 for holding a plurality of inflatable bladders. The control module 114 may be configured to control inflation and deflation of the bladders to apply varying compression pressure to different portions of the body part 200. The user may operate the control module 114 and select predefined compression patterns. The compression apparatus 100 is operable to apply varying compression pressure to different portions of the body part 200, wherein such varying compression pressure may be based on the predefined compression patterns.

The control module 114 may include a processor, storage for the compression patterns, a user interface, and a wireless communication module. The processor may be implemented as a general-purpose processor that is operable to execute processor executable instructions or may be a hard-wired processor. The storage for compression patterns stores data that indicates compression patterns that may be used by the processor to generate control signals. The storage for compression patterns may be implemented as a non-volatile storage. The user interface may be implemented as any form of interface that allows the user to input commands and parameters. For example, the user interface may be implemented as a display and a plurality of input buttons, or a capacitive/touch screen display. The wireless communication module may include one or more of an infrared communication module, a Bluetooth communication module, and a Wi-Fi communication module, which allows the controller to send and receive data and commands from an electronic device or a wireless-enabled device, such as a smartphone device or a computer device. The control module 114 may further include a log memory module to collect and store data collected by the compression apparatus 100 during usage. The data may then be downloaded to the electronic device via the wireless communication module. Alternatively, the compression apparatus 100 may include a data communications port, such as a USB port, for physically connecting to the electronic device to download the collected data.

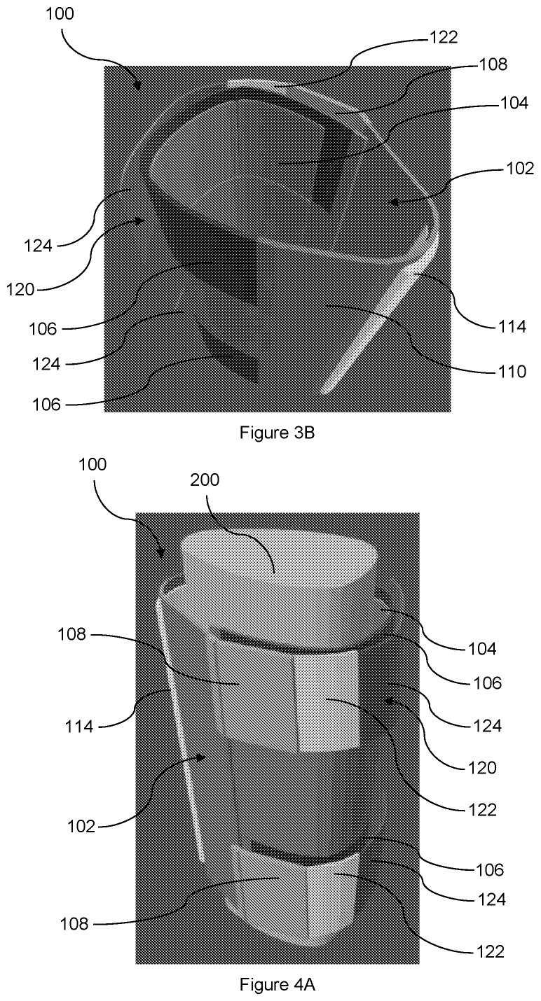

With reference to FIG. 4A to FIG. 4C, after wearing the wearable device 102 and tightening the tensioning device 120, the compression portion 104 applies base compression to the body part 200. The base compression may range from 10 mmHg to 30 mmHg, preferably from 15 mmHg to 20 mmHg, but other ranges are possible as well. Comparatively, the compression apparatus 100 without the tensioning device 120 or without tightening it can achieve a base compression of around 5 to 15 mmHg. The user may then configure the compression portion 104 to apply the main compression to the body part 200, wherein the main compression complements the base compression to increase the overall compression applied to the body part 200. For example, the user selects a preferred compression pattern and the compression portion 104 performs active compression according to the compression pattern. The compression pattern may involve a sequence of compression cycles and each compression cycle may be considered to comprise a compression period followed by a relaxation period. During the compression period, the active compression applied by the compression portion 104 increases the overall compression from the base compression to a peak compression pressure. For example, the active compression may range from 0 mmHg to 20 mmHg and the peak overall compression (active and base compression) may range from 10 mmHg to 40 mmHg. During the relaxation period, the active compression returns to a stable or sustained pressure and the overall compression may be reverted to the initial base compression pressure.

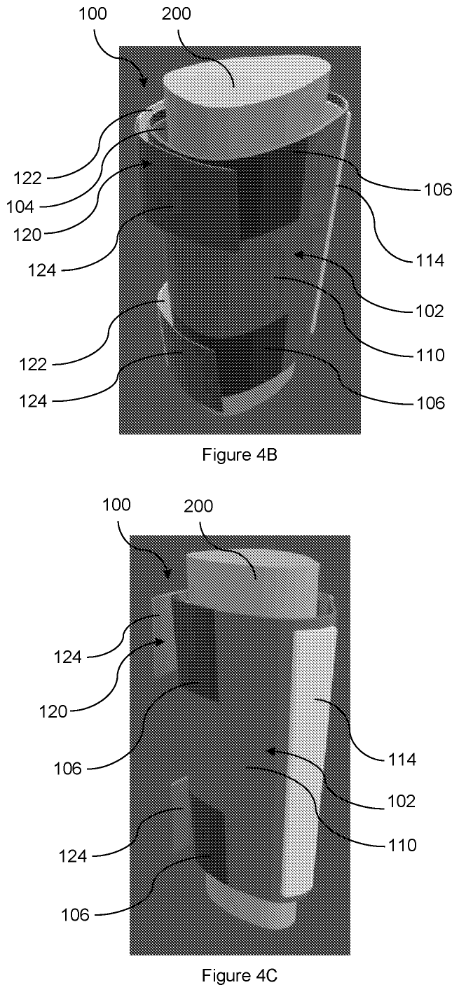

Various tests have been performed to measure the maximum base compression pressures achieved by the compression apparatus 100 at the upper and lower zones of the wearable device 102, such as shown in FIG. 4A to FIG. 4C. The maximum base compression pressures were measured with and without the pre-tension, i.e. with and without the tensioning device 120 or tightening the tensioning device 120. The results of these tests are shown in FIG. 5. Notably, the maximum base compression pressures at the upper and lower zones increased by about 1.5 to 3.5 times when the tensioning device 120 is tightened.

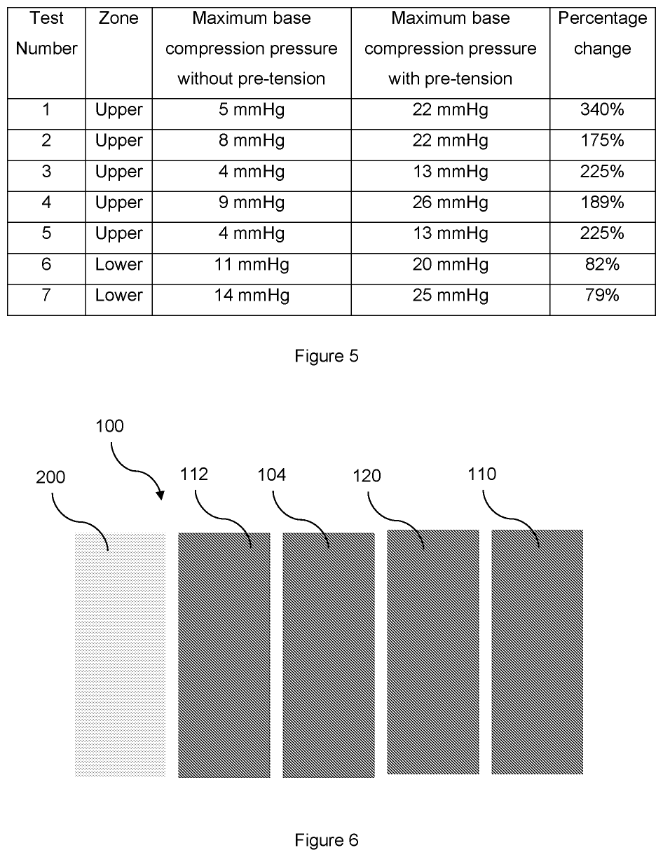

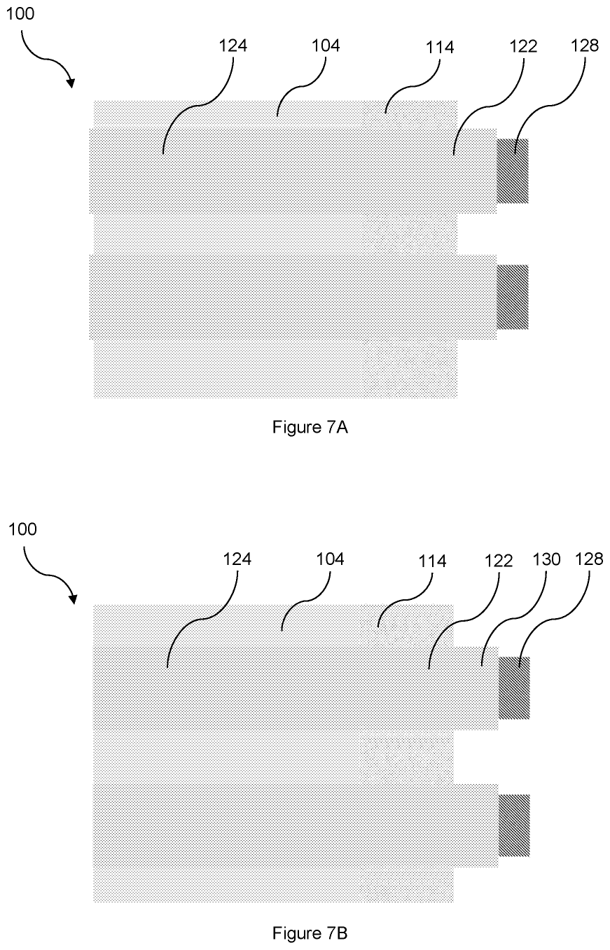

FIG. 6 shows a cross-sectional illustration of the compression apparatus 100 when it is worn on the body part 200. The compression apparatus 100 includes the wearable device 102 (with the compression portion 104) and the tensioning device 120. The compression apparatus 100 also includes the outer layer 110 and skin-contact layer 112 interfacing between the wearable device 102 and the body part 200. With reference to FIG. 7A and FIG. 7B, the wearable device 102 includes the control module 114 attached to the compression portion 104, such that the tensioning device 120 overlays the compression portion 104 and control module 114. The tensioning device 120 includes the elastic portion 122 and inelastic portion 124. The inelastic portion 124 is aligned to the compression portion 104 and the elastic portion 122 is aligned to the control module 114.

In one embodiment as shown in FIG. 7A, the elastic portion 122 is attached to the fastening elements 128 that are attachable to the inelastic portion 124 to tighten the tensioning device 120. Specifically, the fastening elements 128 are attached directly to the elastic portion 122 and are attachable to the opposing end of the inelastic portion 124. In one embodiment as shown in FIG. 7B, the fastening elements 128 are attached to the elastic portion 122 via an intermediary portion 130. The intermediary portion 130 may be an inelastic portion or may include a hybrid of elastic and inelastic portions. It will be appreciated that the tensioning device 120 may include various sections of elastic and inelastic portions in various arrangements. For example, the intermediary portion 130 may include a series of elastic and inelastic portions.

The tensioning device 120 may be integrated with or connected to suitable sensors that can measure the tension level in the tensioning device 120. The measured tension level can be used to determine the level of base compression applied by the compression portion 104 due to the tensioned tensioning device 120. For example, these sensors may be implemented as pressure sensors which directly measure the base compression applied to the body part 200. Alternatively, the sensors may be configured to indirectly measure a quantity from which the applied base compression can be derived or estimated.

Additionally, the compression apparatus 100 may include a visual device cooperative with the tensioning device 120 and configured to inform the user of the base compression level. For example, the visual device may be configured to show colour changes in response to the level of base compression, allowing the user to easily know whether the desired base compression has been achieved. The visual device may include colour-changing touch fabrics and pressure-sensitive elastic materials/fibres integrated with LEDs to visually indicate when the base compression has been reached. The visual device may include colour-changing bands which can indicate the average pressure when a colour is shown in response to stretching of the band.

Despite requiring the user to manually tighten the tensioning device 120 around the body part 200, the user can more accurately tighten the tensioning device 120 to adjust the base compression to the user's desired level. Current static compression devices do not provide such level of compression adjustment for the user. This consequently improves the effectiveness of the compression apparatus 100 in providing compression therapy to the user. The compression apparatus 100 including the wearable device 102 and tensioning device 120 is also easier to use than current active compression devices. More specifically, active compression devices, such as those using inflatable bladders, require the user to operate the devices and inflate the bladders to apply a certain level of base compression. The control interface of such devices may be overly complex for the user and some users may find it difficult to operate these devices, often failing to achieve the desired level of base compression.

Using the tensioning device 120, the user can more easily tighten and loosen it as desired, as opposed to having to fumble through the control interface which can be difficult especially for the elderly.

Instead of being attached to the wearable device 102, the tensioning device 120 may be used as a standalone device to apply compression to the body part 200. More specifically, the tensioning device 120 is wearable around the body part 200 for applying compression to the body part 200. The tensioning device 120 includes one or more straps each having the elastic portion 122 and inelastic portion 124. The elastic portion 122 is attached to one end of the inelastic portion 124 and attachable to the opposing end of the inelastic portion 124. When the tensioning device is worn around the body part 200 and the inelastic portion 124 is arranged on the body part 200, the elastic portion 122 is attached to both ends of the inelastic portion 124 such that the elastic portion 122 is stretched, thereby tightening the tensioning device 120 and causing the inelastic portion 124 to apply compression to the body part 200. As the tensioning device 120 is worn around the body part 200 to apply base compression or pretension, the tensioning device 120 may be referred to as a compression or pretension or pre-compression wrap.

The inelastic portion 124 may be configured for adjustment of an overall length of the inelastic portion 124. For example, the inelastic portion 124 can be worn around the calf area but different people have different sizes of calves. The inelastic portion 124 can be adjustable in length so that the same tensioning device 120 can be used for different people. The inelastic portion 124 may include a plurality of detachably joined sections for adjusting the overall length. More specifically, these sections are detachably joined to each other so that one or more sections can be removed to decrease the overall length of the inelastic portion 124. Conversely, one or more sections can be added to increase the overall length.

This compression apparatus 100 may further include a treatment device such as a compression device, wherein the tensioning device 120 is attachable to the treatment device. The compression device ay be such as an active compression device (e.g. one that uses inflatable bladders) or a static/passive compression device (e.g. a compression sock), wherein the compression device is configured for applying the main compression to the body part 200. The tensioning device 120 is attachable to the compression device and the compression applied by the tensioning device 120 may be referred to as the base compression. The compression device is configured for applying the main compression to the body part 200 in addition to the base compression.

Other than compression devices, the tensioning device 120 can be used with other treatment devices, such as therapeutic devices, to provide some base level of compression cooperative with the core functionality of such devices. For example, such treatment devices can be used for hot/cold therapy, such as thermal devices. Other non-limiting examples of treatment devices include cryotherapy devices, herbal/Ayurvedic devices and wraps, orthopaedic braces and supports, and muscle stimulation devices.

In many embodiments, the tensioning device 120 includes one or more straps each having the elastic portion 122 and inelastic portion 124. In some embodiments, the tensioning device 120 includes wires that are resiliently deformable to tighten the tensioning device 120 around the compression portion 104. For example, the wires are made of or include an electrically-responsive material such that they are resiliently deformable in response to electric current. When an electric current conducts through the wires, the wires constrict and consequently tightens the tensioning device 120. The material of the wires may include a shape memory alloy material. For example, the shape memory alloy material includes a metal alloy of nickel and titanium, such as Nitinol®. Wires made of Nitinol® may be known as muscle wires.

In some embodiments, the tensioning device 120 includes a winding mechanism configured to tighten the tensioning device 120 around the compression portion 104.

The winding mechanism includes a set of wires disposed around the compression portion 104 and a winding component cooperative with the wires. The winding component may be in the form of a dial structure that can be turned to tighten and loosen the wires. The winding mechanism may be similar to various existing fit mechanisms that can be used as a closure mechanism and/or for lacing.

In various embodiments, there may be a structure, such as a garment, that includes the compression apparatus 100. For example, the compression apparatus 100 may be used as a compression garment, or as an accessory to the compression garment, suitable for athletes who require compression for their sports activities. The compression apparatus 100 may also be used in the medical industry, such as compression garments for compression therapy to treat various lymphatic and circulatory conditions. The compression apparatus 100 may alternatively be used in other medical devices like orthopaedic devices.

In the foregoing detailed description, embodiments of the present disclosure in relation to a compression apparatus 100 are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of the present disclosure that a variety of changes and/or modifications can be made to the disclosed embodiments without departing from the scope of the present disclosure. Therefore, the scope of the present disclosure as well as the scope of the following claims is not limited to embodiments described herein.

1. A compression apparatus for applying compression to a body part, the compression apparatus comprising:
a wearable device wearable on the body part, the wearable device comprising a compression portion for applying main compression to the body part; and
a tensioning device attached to the wearable device and wearable on the body part, the tensioning device comprising:
an inelastic portion aligned to the compression portion and for arranging on the body part; and
an elastic portion attached to one end of the inelastic portion,
wherein when the wearable device and tensioning device are worn on the body part, the elastic portion is attachable to an opposing end of the inelastic portion such that the elastic portion is stretched and the tensioning device is tightened around the compression portion, thereby causing the compression portion to apply base compression to the body part; and
wherein the main compression adds to the base compression to increase overall compression applied to the body part, the inelastic portion facilitating even distribution of the overall compression on the body part.

2. The compression apparatus according to claim 1, wherein the elastic portion and the opposing end of the inelastic portion comprise mutually engageable fastening elements.

3. The compression apparatus according to claim 1, wherein the opposing end of the inelastic portion is knitted, stitched, or bonded to the wearable device.

4. The compression apparatus according to claim 1, wherein the elastic portion comprises an elastomeric fabric material.

5. The compression apparatus according to claim 4, wherein the elastomeric fabric material comprises one or more of rubber, polybutadiene, polyisobutylene and polyurethane.

6. The compression apparatus according to claim 1, wherein the inelastic portion comprises a smooth fabric material.

7. The compression apparatus according to claim 6, wherein the smooth fabric material comprises taffeta.

8. The compression apparatus according to claim 1, wherein the tensioning device is removably attached to the wearable device.

9. The compression apparatus according to claim 1, wherein the tensioning device is integrated with the wearable device.

10. The compression apparatus according to claim 1, further including a visual device cooperative with the tensioning device for informing a level of the base compression.

11. The compression apparatus according to claim 1, wherein the wearable device further includes a skin-contact layer for interfacing between the wearable device and the body part, the skin contact layer optionally configured for moisture wicking.

12. The compression apparatus according to claim 11, wherein the skin-contact layer comprises a smooth fabric material.

13. The compression apparatus according to claim 1, wherein the wearable device comprises a control module for controlling the main compression applied by the compression portion.

14. The compression apparatus according to claim 1, wherein the compression portion comprises an inflatable bladder for actively applying the main compression to the body part.

15. The compression apparatus according to claim 1, wherein the inelastic portion is configured for adjustment of an overall length of the inelastic portion.

16. The compression apparatus according to claim 15, wherein the inelastic portion comprises a plurality of detachably joined sections for adjusting the overall length.

17. The compression apparatus according to claim 1, further comprising a treatment device, wherein the tensioning device is attachable to the treatment device.

18. The compression apparatus according to claim 17, wherein the treatment device is a compression device configured for applying the main compression to the body part in addition to the base compression applied by the tensioning device.

19. A garment comprising the compression apparatus according to claim 1.

20. The compression apparatus according to claim 11, wherein the skin-contact layer is configured for moisture wicking.
